(19) United States
(12) Patent Application Publication
(10) Pub. No.: US 2025/0221946 A1
(43) Pub. Date: Jul. 10, 2025

(54) PHARMACEUTICAL COMPOSITION CONTAINING VITAMIN K2 FOR IMPROVING CARDIOVASCULAR CALCIFICATION, PREPARATION METHOD THEREFOR AND USE THEREOF

(71) Applicants: NANJING ASCEND BIOTECHNOLOGY INSTITUTE CO., LTD., Nanjing (CN); NANJING YISHENG KANGPIN BIOTECHNOLOGY CO., LTD., Nanjing (CN)

(72) Inventors: Tianming HE, Nanjing (CN); Luqiu SHI, Nanjing (CN); Hongyu XUE, Nanjing (CN); Guizhen SU, Nanjing (CN)

(73) Assignees: NANJING ASCEND BIOTECHNOLOGY INSTITUTE CO., LTD., Nanjing, JS (CN); NANJING YISHENG KANGPIN BIOTECHNOLOGY CO., LTD., Nanjing, JS (CN)

(21) Appl. No.: 18/850,585

(22) PCT Filed: Apr. 29, 2022
(86) PCT No.: PCT/CN2022/090653

§ 371 Date: Sep. 25, 2024

(30) Foreign Application Priority Data
Mar. 25, 2022 (CN) 202210302888.9

(51) Int. Cl. A61K 31/122; A61K 9/50; A61K 31/202; A61K 31/4745; A61K 31/593; A61P 9/10

(52) U.S. Cl. CPC A61K 31/122; A61K 9/5084; A61K 9/5089; A61K 31/202; A61K 31/4745; A61K 31/593; A61P 9/10

(57) ABSTRACT

A vitamin K₂-containing pharmaceutical composition for improving cardiovascular calcification is provided. The pharmaceutical composition includes a first component, a second component and a third component, with the mass ratio of (0.1-5):(0.01-0.5):(100-500). The preparation method and use of the pharmaceutical composition are also provided. The pharmaceutical composition can comprehensively prevent and block cardiovascular calcification, and is particularly suitable for reversing the cardiovascular calcification symptom due to taking statin drugs. Meanwhile, the pharmaceutical composition can provide daily health support and protection for the heart. The pharmaceutical composition of the present disclosure is characterized by stable active ingredients, high bioavailability, strong targeting and no side effect, achieving the purpose of prevention and treatment simultaneously.

### TECHNICAL FIELD

The present disclosure relates to the technical fields of pharmaceuticals, health products and foods, and in particular to a vitamin K₂-containing pharmaceutical composition for improving cardiovascular calcification, and preparation method and use thereof.

### BACKGROUND OF THE INVENTION

Vascular calcification refers to inappropriate biological calcification in the soft tissue of cardiovascular system, which is considered as a pathological change of calcium salt depositing in arterial wall tissue. It is commonly seen in atherosclerosis, diabetes, nephropathy, aging, systolic hypertension and many other diseases, which can easily lead to myocardial infarction and stroke. Previous studies have shown that the vascular calcification is a passive, degenerative and inevitable terminal process. However, the recent clinical and basic study results have shown that the vascular calcification is an active, adjustable, treatable and preventable process similar to the process of bone development and cartilage formation.

Coronary heart disease is considered as the healthy and life main cutthroat for middle-aged and elderly people in today's society. With the progress of society and the improvement of civilization, the incidence of coronary heart disease is rising year by year, with a tendency to young people. At present, nearly 200 million people in the world are using statin drugs to prevent and treat the coronary heart disease. The latest study has shown that the people who use statin drugs at high frequency are more likely to have promoted coronary artery calcification than those using statin drugs at low frequency.

Based on the existing pharmacological evidence and clinical trial results, the main physiological mechanism by which “statin” drugs promote the arterial calcification is as follows: First, inhibit the function of vitamin K which causes arterial calcium deposition; second, inhibit the synthesis of coenzyme Q₁₀ in the body which leads to the decrease of the level of anti-oxidative coenzyme Q₁₀, and damage the energy metabolism of mitochondria which leads to the sharp decline of the body's anti-oxidative capacity, thus inducing myocardial injury.

Therefore, completely blocking and preventing cardiovascular calcification is an important factor to ensure the health of those people who are using statin drugs, reduce the side effects and improve the quality of life.

At present, vitamin K₂ is generally used as a dietary supplement to promote the calcium absorption, protect the joint health and strengthen the bones. However, its function of reversing calcification has not been heeded or is rarely used. Meanwhile, due to that vitamin K₂ is a fat-soluble vitamin with low solubility in water, it is difficult to be absorbed by intestine after oral administration, and it is low in terms of bioavailability. Moreover, vitamin K₂ is extremely sensitive to light and heat, the shelf life of conventional-related products is unstable, and its biological activity is seriously reduced after long-term storage. Therefore, vitamin K₂-related products with stable activity, high bioavailability and functional targeting are urgently needed.

### SUMMARY OF THE INVENTION

The objective of the present disclosure is to overcome the above shortcomings in existing technology and provide a vitamin K₂-related product with stable activity, high bioavailability and functional targeting.

In order to achieve the above objective, the present disclosure provides the following technical solution.

In one aspect, the present disclosure provides a vitamin K₂-containing pharmaceutical composition for improving cardiovascular calcification, where the pharmaceutical composition includes first component, second component and third component, where the first component includes active functional ingredients promoting calcium absorption, the second component includes active functional ingredients inhibiting or reversing calcification and the third component includes active functional ingredients providing daily health protection for cardiovascular system;

The mass ratio of first component, second component and third component is (0.1-5):(0.01-0.5):(100-500).

In some embodiments, the first component includes active ingredients promoting calcium absorption, where the active ingredients include main active ingredient and auxiliary active ingredient, where the main active ingredient is vitamin D₃ and the auxiliary active ingredient is one or more of lactose, vitamin A, albumin polypeptide, vitellin peptide, fructooligosaccharide, galactooligosaccharide and β-carotene.

In other some embodiments, the second component includes active ingredients inhibiting or reversing calcification, where the active ingredients include main active ingredient and auxiliary active ingredient, where the main active ingredient is vitamin K₂ and the auxiliary active ingredient is one or more of naringin, arachidonic acid, tripterine, puerarin, apigenin and aconite polysaccharide.

In another some embodiments, the third component includes active ingredients providing daily health protection for cardiovascular system, where the active ingredients include main active ingredient and auxiliary active ingredient, where the main active ingredient is Ω-3 fatty acid, pyrroloquinoline quinone and coenzyme Q₁₀, and the auxiliary active ingredient is one or more of epicatechin, pumpkin seed oil, phospholipid, krill oil, spirulina, quercetin, lycopene, pomegranate polyphenols, resveratrol, cyanidin, soy isoflavones and allicin.

Preferably, the first component accounts for 0.1%-1% based on the total mass of the pharmaceutical composition; the second component accounts for 0.001%-0.1% based on the total mass of the pharmaceutical composition; the third component accounts for 30%-85% based on the total mass of the pharmaceutical composition.

In addition, preferably, the pharmaceutical composition also includes auxiliary, where the auxiliary is one or more of microcrystalline cellulose, silicon dioxide, magnesium stearate, α-cyclodextrin, mannitol, sodium carboxymethyl cellulose, hydroxy propyl cellulose, glycerol, gelatin, methyl cellulose, ethyl cellulose, hydroxypropyl methyl cellulose, talcum powder, modified starch, antioxidant, titanium dioxide, tartrazine aluminum lake, sunset yellow aluminum lake, carmine aluminum lake, brown iron oxide, glyceryl triacetate, polyethylene glycol, cross-linked povidone and cross-linked sodium carboxymethyl cellulose.

More preferably, the pharmaceutical composition includes the main ingredients in different parts by mass, as follows: Vitamin K₂ as 0.01-1 parts, Ω-3 fatty acid as 300-3,000 parts, pyrroloquinoline quinone as 1-40 parts, coenzyme Q₁₀ as 10-500 parts and vitamin D 3 as 1-60 parts.

In more preferable embodiments, the vitamin K₂ is in the form of MK-7 with all-trans structure; the ratio of EPA to DHA in the Ω-3 fatty acid is (0.1-4): 1.

In another aspect, the present disclosure also provides the use of the pharmaceutical composition in dietary supplements and health care products.

In the third aspect, the present disclosure provides a soft capsule that includes any of vitamin K₂-containing pharmaceutical compositions for improving cardiovascular calcification.

In the preferable embodiments, the soft capsule includes the main ingredients in different parts by mass, as follows: Vitamin K₂ as 0.01-0.7 parts, Ω-3 fatty acid as 500-2,500 parts, pyrroloquinoline quinone as 1-30 parts, coenzyme Q₁₀ as 50-500 parts, vitamin D₃ as 1-50 parts, soft capsule skin as 50-500 parts, enteric coating powder as 10-100 parts and auxiliary as 20-200 parts.

In the fourth aspect, the present disclosure also provides the preparation method of the soft capsule, including the following steps:
(1) Granulating: Under the dark condition, evenly mix the fat-insoluble ingredients in the second component and the third component with some auxiliaries and make granulation by dry method, where the particle size is controlled as 50-200 meshes, in order to obtain granulated particles;
(2) Tabletting: Use the tablet machine to make the particles obtained in Step (1) into naked tablets with a specified weight;
(3) Coating tablet: Mix the naked tablets obtained in Step (2) with the enteric coating powder to obtain the coating tablets according to the normal coating steps;
4) Capsule liquid: Weigh and take the fat-soluble ingredients in the third component, the first component and the antioxidant in the auxiliary by weight, dissolve the antioxidant and the first component into the oil with fat-soluble ingredients in the third component respectively at a temperature of 20-60° C. to obtain the mixed oil liquid, and then let it stand for 4-12 h to obtain the capsule liquid;
(5) Soft capsule skin: Obtain the soft capsule skins by adopting the general formula and preparation method in this industry;
(6) Soft capsule: Inject the capsule liquid obtained in Step (4) between the two layers of capsule skins containing the coating tablet obtained in Step (3), make pelleting, and at last obtain the soft capsules internally containing the tablet.

Compared with the existing technology, the present disclosure has the following beneficial effects:
(1) Blocking and preventing cardiovascular calcification in a targeted way: In one aspect, promote the calcium absorption, and avoid the accumulation of excess calcium on the blood vessel wall; in another aspect, strengthen the conversion of incorrectly deposited calcium; in the third aspect, strengthen the energy metabolism of mitochondria to improve the myocardial capacity, enhance the resistance and avoid the body injury, thereby providing prevention in advance at the macro level, blocking the risk source and providing pre-determined protection measures. While improving vascular calcification, it can also prevent and eliminate other risks caused by vascular calcification;
(2) In terms of the product obtained by adopting this preparation method, due to special dual-protection mode, it avoids not only the influences from light, heat, acid and other environments as well as the first-pass effect of oral cavity, intestines and stomach, but also the premature decomposition after ingestion. Therefore, the activity of MK-7 and other functional components in the composition is more stable and long-lasting, the bioavailability is higher, the product quality is more stable and the shelf life is longer;
(3) While improving calcification, it is also characterized by body immunity improvement, anti-oxidation, cancer prevention, anti-aging and bone strengthening.

### BRIEF DESCRIPTION OF THE DRAWINGS

FIG. 1 shows the trend regarding the influence of pharmaceutical composition on OPG (osteoprotegerin) level in rat serum.

FIG. 2 shows the trend regarding the influence of pharmaceutical composition on OPN (osteopontin) level in rat serum.

FIG. 3 shows the trend regarding the influence of pharmaceutical composition on CaBP (calcium binding protein) level in rat serum.

FIG. 4 shows the trend regarding the influence of pharmaceutical composition on BMP-7 (bone morphogenetic protein-7) level in rat serum.

FIG. 5 shows the trend regarding the influence of pharmaceutical composition on calcium content level in rat's artery blood vessel.

### DETAILED DESCRIPTION OF THE INVENTION

The technical solutions in the embodiments of the present disclosure are clearly and completely described in conjunction with the accompanying drawings in the embodiments of the present disclosure. It is understood that the described embodiments are only a part of the embodiments of the present disclosure, instead of all embodiments. All other embodiments, which can be derived by those skilled in the art from the embodiments given herein without making any creative effort, shall fall within the protection scope of the present disclosure.

The present disclosure provides a vitamin K₂ (MK-7)-containing pharmaceutical composition for improving cardiovascular calcification, and preparation method and use thereof. The pharmaceutical composition can comprehensively prevent and block cardiovascular calcification, and is particularly suitable for reversing the cardiovascular calcification symptom due to taking statin drugs. Meanwhile, the pharmaceutical composition can provide daily health support and protection for the heart.

The present disclosure has the effect of improving cardiovascular calcification from three aspects: Promotion of calcium absorption in a targeted way, reversion of calcification and daily protection. In one aspect, promote the calcium absorption, and avoid the improper accumulation of calcium on the blood vessel wall; in another aspect, strengthen the conversion of incorrectly deposited calcium; in the third aspect, strengthen the energy metabolism of mitochondria to improve the myocardial capacity, enhance the resistance and avoid the body injury, thereby providing prevention in advance at the macro level, blocking the risk source and providing pre-determined protection measures. With the synergy from three aspects, an overall improvement for cardiovascular calcification can be achieved.

In one aspect, the present prevention provides a vitamin K₂-containing pharmaceutical composition for improving cardiovascular calcification. The pharmaceutical composition includes first component, second component and third component,

The mass ratio of first component, second component and third component is (0.1-5): (0.01-0.5):(100-500).

The first component includes active ingredients promoting calcium absorption, which may include one or more of lactose, vitamin A, vitamin D₃, albumin polypeptide, vitellin peptide, fructooligosaccharide, galactooligosaccharide and β-carotene.

As one of the D vitamins, vitamin D₃, also known as cholecalciferol, can improve the absorption of calcium and phosphorus by the body and make the levels of plasma calcium and plasma phosphorus reach saturation point. Studies have shown that, as the precursor of D hormone, vitamin D₃ needs to be hydroxylated in kidneys and lungs to become active vitamin D which can promote the reabsorption of calcium in kidneys and lungs. Therefore, the present disclosure preferably compounds VD₃ and MK-7, which can promote the absorption of calcium and avoid excessive deposition of absorbed calcium in one aspect, and give a full play of MK-7 to activate osteocalcin serving as calcium claw to grab the deposited calcium into bones in order to block and prevent calcification in another aspect.

Preferably, vitamin D₃ is used as the main ingredient of the first component in the present disclosure.

More preferably, vitamin D₃ in the present disclosure is plant-based, with the effective active unit≥400 IU.

The second component includes active ingredients inhibiting or reversing calcification, which may include one or more of naringin, arachidonic acid, tripterine, puerarin, apigenin, aconite polysaccharide and vitamin K₂.

As a kind of fat-soluble vitamin, vitamin K₂ is mainly used to treat and prevent osteoporosis. According to the length of tail chain, K₂ can be divided into MK-4, MK-7, MK-8, MK-10 and other sub-categories. All the K₂ vitamins are similar in structure, with different side chain lengths. The longer the side chain, the better the absorption, the higher the biological activity, and the longer it exists in the blood. Therefore, long-chained MK-7 is considered as the best, due to that it is proved to have the highest bioavailability and a long half-life after oral administration, allowing it to exert its benefits over a longer period of time. In addition, MK-7 is obtained by plant fermentation, which is considered as more healthy.

As a protein secreted by osteoblast, osteocalcin, also known as bone γ-carboxyglutamic acid protein, is a vitamin K-dependent protein which is regarded as the specific executor of introducing calcium into bones. When it is first secreted, osteocalcin has no physiological activity and must undergo carboxylation to exert its physiological effects. After carboxylation, osteocalcin will be able to firmly grab calcium salt, promote the deposition of calcium salt in the bones, and improve the bone mineralization rate. Therefore, vitamin K₂ is crucial for maintaining bone health and preventing osteoporosis. In case that the body is short of vitamin K₂, osteocalcin will not form calcium claw, just like a human hand without a thumb, causing decreased grab ability. As a consequence, a large amount of calcium will be lost from the bones, thus leading to osteoporosis.

In addition to participating in normal body functions and metabolism, most of calcium ions in the blood have two different destinations: (1) Depositing at the correct parts, such as bones and teeth; (2) Depositing at the incorrect parts, including articular cartilage, cardiovascular and cerebrovascular arteries, gall bladder and kidney and other soft tissues. Incorrectly deposited calcium will cause functional damage to the tissues and organs at the parts where the calcium is deposited. If most of the blood calcium cannot migrate to bones, it will be heavily deposited in soft tissues such as blood vessel. Long-term accumulation will cause organ calcification, the most common of which is vascular calcification. However, the body has a mechanism to prevent this abnormal deposition. Many studies have shown that vitamin K₂ is able to prevent the incorrect deposition of calcium. Carboxylated matrix γ-carboxyglutamic acid protein can prevent calcium from depositing in soft tissues such as blood vessels and cartilage, and can bind with calcium ions deposited on blood vessel walls to remove them from blood vessels, thus improving arterial calcification.

Therefore, the present disclosure preferably utilizes activating osteocalcin and accurately grabbing incorrectly deposited calcium by vitamin K₂ as the core point of difference, in order to block and prevent cardiovascular calcification in a targeted way.

More preferably, the vitamin K₂ is MK-7 structured, and obtained by organic bean fermentation method. Compared with chemical synthesis method, the vitamin K₂ is characterized by no organic reagent residue, green in the whole process and being safer to take;

Further preferably, the MK-7 is in the form of all-trans structure, with purity≥99.5%. The MK-7 with all-trans structure has a higher bioavailability.

The third component includes active ingredients providing daily health protection for cardiovascular system, which may include one or more of Ω-3 fatty acid, pyrroloquinoline quinone (PQQ), coenzyme Q₁₀ (CoQ₁₀), epicatechin, pumpkin seed oil, phospholipid, krill oil, spirulina, quercetin, lycopene, pomegranate polyphenols, resveratrol, cyanidin, soy isoflavones and allici.

As a kind of fat-soluble quinone with a structure similar to vitamin K, the coenzyme Q₁₀ is a quinone ring compound which is named due to that the polymerization degree of side chain-polyisoprenyl group at the position of the sixth carbon atom of its parent nucleus is 10. Studies have shown that the cholesterol-lowering statin drugs can reduce the level of coenzyme Q₁₀ in the body by as much as 40%, making heart problems more serious. While we are taking statin drugs, the coenzyme Q₁₀ shall be taken in a supplementary manner to reduce the side effects of statin drugs, relieve muscle pain and fatigue caused by drugs, and protect the liver. Meanwhile, the coenzyme Q₁₀ can activate human cells and generate energy, with the functions such as improving immunity, enhancing anti-oxidative capacity, delaying aging, and strengthening human vitality.

PQQ, produced by Gram-negative bacteria, provides extensive nutrition for microorganisms, animals and plants, and possesses anti-oxidative nutrient elements. The protective effect of PQQ on the heart is related to its ability to scavenge free radicals. PQQ can remove the reactive oxygen produced by anoxia-reperfusion and significantly reduce the release of lactate dehydrogenase in the heart. Under the catalysis of flavin reductase, its catalytic products can reduce the peroxide state of hemoglobin and eliminate the damage to myocardium caused by anoxia-reperfusion. Studies have shown that using PQQ to protect the heart of ischemia-reperfusion mice can significantly reduce the myocardial infarction size, enhance the rising and falling rate of left ventricular pressure and left ventricular diastolic pressure, reduce ventricular fibrillation, and lower the level of malondialdehyde in myocardial tissues. PQQ can also inhibit the production of ROS in rat cardiomyocytes induced by hydrogen peroxide and reduce the mitochondrial membrane potential, thus reducing oxidative stress, inhibiting the inactivation of mitochondrial function and protecting rat cardiomyocytes.

Therefore, in terms of the characteristic that the level of coenzyme Q₁₀ in the body will be reduced after taking statin drugs for a long time, the present disclosure preferably compounds PQQ and CoQ10 to enhance the level of coenzyme Q₁₀ in the body in one aspect, and activate the cells, strengthen the energy metabolism of mitochondria, improve the myocardial capacity and avoid damage caused by drugs in another aspect. Both PQQ and CoQ₁₀ can improve the body's anti-oxidative capacity and maintain the free radical-scavenging balance.

Preferably, PQQ can be in the disodium salt form of pyrroloquinoline quinone or in the acid form of pyrroloquinoline quinone, with purity≥99%.

Preferably, CoQ10's purity≥98%.

As a long-chain and polyunsaturated fatty acid, Ω-3 is discovered from the plants and marine organisms. The existing study data shows that Ω-3 has the efficacy of promoting heart health and preventing coronary artery diseases. Ω-3 can mainly protect the inner wall cells of blood vessels, restore vascular elasticity, dilate blood vessels, inhibit platelet aggregation, lower blood lipids, decrease blood pressure and inhibit the thrombus formation, thus effectively preventing and treating cardiovascular and cerebrovascular diseases.

Moreover, Ω-3 can prevent arrhythmia and sudden cardiac death. A clinical study on more than 11,000 patients with coronary heart disease has shown that taking 1-2 grams of Ω-3 unsaturated fatty acid every day can significantly reduce the death rate of patients with cardiovascular diseases, and especially reduce the incidence rate of sudden cardiac death by up to 45%.

There are mainly three kinds of Ω-3 fatty acids, including α-linolenic acid, eicosapentaenoic acid (EPA) and docosahexaenoic acid (DHA). EPA can prevent stroke or myocardial infarction, lower blood cholesterol and prevent arteriosclerosis. DHA, with the function of activating brain cells, can promote and coordinate the nerve circuit conduction, and maintain the normal operation of brain cells. DHA supplementation can improve concentration loss, learning disabilities, memory loss and senile dementia.

Meanwhile, Ω-3 can also fight nervous system diseases, prevent and fight cancers, and fight inflammation. Therefore, using Ω-3 supplements as a part of lifestyle intervention in the pharmaceutical composition of the present disclosure can reduce the death rate and the risk of sudden cardiac death.

Preferably, in the present disclosure, the ratio of EPA to DHA in the Ω-3 fatty acid is (0.1-4): 1;

More preferably, EPA content in the Ω-3 fatty acid≥40%;

Further preferably, EPA content in the Ω-3 fatty acid≥45%.

The present disclosure has found that there are many active ingredients or dietary supplements in this field that can comprehensively block and prevent cardiovascular calcification, but the synergistic effect cannot be simply achieved by compounding. Therefore, according to the mechanism of calcification, the features of cardiovascular diseases and the metabolic characteristics of statin drugs, the ingredients are screened out for compounding through numerous tests, which can promote the calcium absorption and avoid the accumulation of excess calcium on the blood vessel wall in one aspect, strength the conversion of incorrectly deposited calcium in another aspect and strengthen the energy metabolism of mitochondria to improve the myocardial capacity, enhance the resistance and avoid the body injury, thereby providing prevention in advance at the macro level, blocking the risk source and providing pre-determined protection measures. While improving vascular calcification, it can also prevent and eliminate other risks caused by vascular calcification.

As one of the specific embodiments of the present disclosure, the vitamin K₂ (MK-7)-containing pharmaceutical composition for improving cardiovascular calcification includes the following ingredients by mass: Vitamin K₂ (MK-7) as 0.01-1 parts, Ω-3 fatty acid as 300-3,000 parts, pyrroloquinoline quinone (PQQ) as 1-40 parts, coenzyme Q₁₀ (CoQ₁₀) as 10-500 parts and vitamin D₃ (VD₃) as 1-60 parts.

In a more preferred embodiment, the pharmaceutical composition also includes auxiliary which is essential for preparing the vitamin K₂ (MK-7)-containing pharmaceutical composition for improving cardiovascular calcification provided by the present disclosure, including coating powder, antioxidant, lubricant and capsule skin. More preferably, the auxiliary is one or more of microcrystalline cellulose, silicon dioxide, magnesium stearate, α-cyclodextrin, mannitol, sodium carboxymethyl cellulose, hydroxy propyl cellulose, glycerol, gelatin, methyl cellulose, ethyl cellulose, hydroxypropyl methyl cellulose, talcum powder, modified starch, antioxidant, titanium dioxide, tartrazine aluminum lake, sunset yellow aluminum lake, carmine aluminum lake, brown iron oxide, glyceryl triacetate, polyethylene glycol, cross-linked povidone and cross-linked sodium carboxymethyl cellulose.

The dosage form of the pharmaceutical composition is one or more of tablets, powders, hard capsules, soft capsules, and suspensions.

In the second aspect, the present disclosure provides the use of vitamin K₂-containing pharmaceutical composition for improving cardiovascular calcification in dietary supplements and health care products.

In the third aspect, the present disclosure provides a soft capsule that includes the vitamin K₂-containing pharmaceutical composition for improving cardiovascular calcification. Preferably, the soft capsule includes the ingredients in different parts by mass, as follows: Vitamin K₂ (MK-7) as 0.01-0.7 parts, Ω-3 fatty acid as 500-2,500 parts, pyrroloquinoline quinone (PPQ) as 1-30 parts, coenzyme Q₁₀ (CoQ₁₀) as 50-500 parts, vitamin D₃ (VD₃) as 1-50 parts, soft capsule skin as 50-500 parts, enteric coating powder as 10-100 parts and auxiliary as 20-200 parts.

In the fourth aspect, the present disclosure provides the preparation method of the vitamin K₂ (MK-7)-containing pharmaceutical composition for improving cardiovascular calcification. The preparation method is as follows:
(1) Granulating: Under the dark condition, evenly mix the fat-insoluble ingredients in the second component and the third component with some auxiliaries and make granulation by dry method, where the particle size is controlled as 50-200 meshes, in order to obtain granulated particles;
(2) Tabletting: Use the tablet machine to make the particles obtained in Step (1) into naked tablets with a specified weight;
(3) Coating tablet: Mix the naked tablets obtained in Step (2) with the enteric coating powder to obtain the coating tablets according to the normal coating steps;
(4) Capsule liquid: Weigh and take the fat-soluble ingredients in the third component, the first component and the antioxidant in the auxiliary by weight, dissolve the antioxidant and the first component into the oil with fat-soluble ingredients in the third component respectively at a temperature of 20-60° C. to obtain the mixed oil liquid, and then let it stand for 4-12 h to obtain the capsule liquid;
(5) Soft capsule skin: Obtain the soft capsule skins by adopting the general formula and preparation method in this industry;
(6) Soft capsule: Adopt the specialized equipment to inject the capsule liquid obtained in Step (4) between the two layers of capsule skins containing the coating tablet obtained in Step (3), make pelleting by special molds, and at last obtain the soft capsules internally containing the tablet

The preparation method of the present disclosure also includes further processing method, including but not limited to drying, pill cleaning, pill picking, packaging, etc., and finally obtaining the finished product.

In terms of the product obtained by adopting this preparation method, due to special dual-protection mode, it avoids not only the influences from light, heat, acid and other environments as well as the first-pass effect of oral cavity and stomach, but also the premature decomposition after ingestion. Therefore, the activity of MK-7 and other active components in the composition is more stable and long-lasting, the bioavailability is higher, the product quality is more stable and the shelf life is longer.

In the third aspect, the present disclosure provides the use of vitamin K₂ (MK-7)-containing pharmaceutical composition for improving cardiovascular calcification in dietary supplements and health care products.

The following embodiments are used to illustrate the present disclosure but do not limit its scope.

### Embodiment 1

This embodiment provides a vitamin K₂ (MK-7)-containing pharmaceutical composition for improving cardiovascular calcification, which includes, by weight parts, the following ingredients:

Vitamin K₂ (MK-7) as 0.18 parts, Ω-3 fatty acid as 1,200 parts, PQQ as 10 parts, coenzyme Q₁₀ (CoQ₁₀) as 200 parts, vitamin D₃ (VD₃) as 5 parts, soft capsule skin as 250 parts, enteric coating powder as 100 parts and auxiliary as 50 parts.

The preparation method is as follows:
(1) Granulating: Under the dark condition, mix MK-7, PQQ and tabletting auxiliary in equal proportion, and make granulation by dry method, where the particle size is controlled as 100 meshes, in order to obtain granulated particles;
(2) Tabletting: Use the tablet machine to make the particles obtained in Step (1) into naked tablets with a specified weight as 0.2 g;
(3) Coating tablet: Mix the naked tablets obtained in Step (2) with the enteric coating powder to obtain the coating tablets according to the normal coating steps;
(4) Capsule liquid: Weigh and take 22-3, CoQ₁₀, VD₃ and antioxidant by weight, dissolve the antioxidant, CoQ₁₀ and VD₃ into Ω-3 oil respectively at a temperature of 40° C. to obtain the mixed oil liquid, and then let it stand for 12 h to obtain the capsule liquid;
(5) Soft capsule skin: Obtain the soft capsule skins by adopting the general formula and preparation method in this industry;
(6) Soft capsule: Adopt the specialized equipment to inject the capsule liquid obtained in Step (4) between the two layers of capsule skins containing the coating tablet obtained in Step (3), make pelleting by special molds, and at last obtain the soft capsules internally containing the tablet.

### Embodiment 2

This embodiment provides a vitamin K₂ (MK-7)-containing pharmaceutical composition for improving cardiovascular calcification, which includes, by weight parts, the following ingredients:

Vitamin K₂ (MK-7) as 0.09 parts, Ω-3 fatty acid as 600 parts, PQQ as 10 parts, coenzyme Q₁₀ (CoQ₁₀) as 100 parts, vitamin D₃ (VD₃) as 2 parts, soft capsule skin as 150 parts, enteric coating powder as 80 parts and auxiliary as 30 parts.

The preparation method is as follows:
(1) Granulating: Under the dark condition, mix MK-7, PQQ and tabletting auxiliary in equal proportion, and make granulation by dry method, where the particle size is controlled as 120 meshes, in order to obtain granulated particles; (2) Tabletting: Use the tablet machine to make the particles obtained in Step (1) into naked tablets with a specified weight as 0.15 g;
(2) Tabletting: Use the tablet machine to make the particles obtained in Step (1) into naked tablets with a specified weight as 0.15 g;
(3) Coating tablet: Mix the naked tablets obtained in Step (2) with the enteric coating powder to obtain the coating tablets according to the normal coating steps;
(4) Capsule liquid: Weigh and take Ω-3, CoQ₁₀, VD₃ and antioxidant by weight, dissolve the antioxidant, CoQ₁₀ and VD₃ into Ω-3 oil respectively at a temperature of 40° C. to obtain the mixed oil liquid, and then let it stand for 12 h to obtain the capsule liquid;
(5) Soft capsule skin: Obtain the soft capsule skins by adopting the general formula and preparation method in this industry;
(6) Soft capsule: Adopt the specialized equipment to inject the capsule liquid obtained in Step (4) between the two layers of capsule skins containing the coating tablet obtained in Step (3), make pelleting by special molds, and at last obtain the soft capsules internally containing the tablet.

### Embodiment 3

This embodiment provides a vitamin K₂ (MK-7)-containing pharmaceutical composition for improving cardiovascular calcification, which includes, by weight parts, the following ingredients:

Vitamin K₂ (MK-7) as 0.12 parts, Ω-3 fatty acid as 900 parts, PQQ as 15 parts, coenzyme Q₁₀ (CoQ₁₀) as 150 parts, vitamin D₃ (VD₃) as 3 parts, soft capsule skin as 180 parts, enteric coating powder as 70 parts and auxiliary as 40 parts.

The preparation method is as follows:
(1) Granulating: Under the dark condition, mix MK-7, PQQ and tabletting auxiliary in equal proportion, and make granulation by dry method, where the particle size is controlled as 80 meshes, in order to obtain granulated particles;
(2) Tabletting: Use the tablet machine to make the particles obtained in Step (1) into naked tablets with a specified weight as 0.25 g;
(3) Coating tablet: Mix the naked tablets obtained in Step (2) with the enteric coating powder to obtain the coating tablets according to the normal coating steps;
(4) Capsule liquid: Weigh and take Ω-3, CoQ₁₀, VD₃ and antioxidant by weight, dissolve the antioxidant, CoQ₁₀ and VD₃ into Ω-3 oil respectively at a temperature of 50° C. to obtain the mixed oil liquid, and then let it stand for 12 h to obtain the capsule liquid;
(5) Soft capsule skin: Obtain the soft capsule skins by adopting the general formula and preparation method in this industry;
(6) Soft capsule: Adopt the specialized equipment to inject the capsule liquid obtained in Step (4) between the two layers of capsule skins containing the coating tablet obtained in Step (3), make pelleting by special molds, and at last obtain the soft capsules internally containing the tablet.

### Embodiment 4

This embodiment provides a vitamin K₂ (MK-7)-containing pharmaceutical composition for improving cardiovascular calcification, which includes, by weight parts, the following ingredients:

Vitamin K₂ (MK-7) as 0.45 parts, Ω-3 fatty acid as 1,000 parts, PQQ as 12 parts, coenzyme Q₁₀ (CoQ₁₀) as 250 parts, vitamin D₃ (VD₃) as 4 parts, soft capsule skin as 180 parts, enteric coating powder as 90 parts and auxiliary as 45 parts.

The preparation method is as follows:
(1) Granulating: Under the dark condition, mix MK-7, PQQ and tabletting auxiliary in equal proportion, and make granulation by dry method, where the particle size is controlled as 100 meshes, in order to obtain granulated particles;
(2) Tabletting: Use the tablet machine to make the particles obtained in Step (1) into naked tablets with a specified weight as 0.26 g;
(3) Coating tablet: Mix the naked tablets obtained in Step (2) with the enteric coating powder to obtain the coating tablets according to the normal coating steps;
(4) Capsule liquid: Weigh and take 22-3, CoQ₁₀, VD₃ and antioxidant by weight, dissolve the antioxidant, CoQ₁₀ and VD₃ into Ω-3 oil respectively at a temperature of 45° C. to obtain the mixed oil liquid, and then let it stand for 8 h to obtain the capsule liquid;
(5) Soft capsule skin: Obtain the soft capsule skins by adopting the general formula and preparation method in this industry;
(6) Soft capsule: Adopt the specialized equipment to inject the capsule liquid obtained in Step (4) between the two layers of capsule skins containing the coating tablet obtained in Step (3), make pelleting by special molds, and at last obtain the soft capsules internally containing the tablet.

### Embodiment 5

This embodiment provides a vitamin K₂ (MK-7)-containing pharmaceutical composition for improving cardiovascular calcification, which includes, by weight parts, the following ingredients:

Vitamin K₂ (MK-7) as 0.65 parts, Ω-3 fatty acid as 1,300 parts, PQQ as 20 parts, coenzyme Q₁₀ (CoQ₁₀) as 200 parts, vitamin D₃ (VD₃) as 5 parts, soft capsule skin as 300 parts, enteric coating powder as 100 parts and auxiliary as 50 parts.

The preparation method is as follows:
(1) Granulating: Under the dark condition, mix MK-7, PQQ and tabletting auxiliary in equal proportion, and make granulation by dry method, where the particle size is controlled as 120 meshes, in order to obtain granulated particles;
(2) Tabletting: Use the tablet machine to make the particles obtained in Step (1) into naked tablets with a specified weight as 0.3 g;
(3) Coating tablet: Mix the naked tablets obtained in Step (2) with the enteric coating powder to obtain the coating tablets according to the normal coating steps;
(4) Capsule liquid: Weigh and take Ω-3, CoQ₁₀, VD₃ and antioxidant by weight, dissolve the antioxidant, CoQ₁₀ and VD₃ into Ω-3 oil respectively at a temperature of 40° C. to obtain the mixed oil liquid, and then let it stand for 10 h to obtain the capsule liquid;
(5) Soft capsule skin: Obtain the soft capsule skins by adopting the general formula and preparation method in this industry;
(6) Soft capsule: Adopt the specialized equipment to inject the capsule liquid obtained in Step (4) between the two layers of capsule skins containing the coating tablet obtained in Step (3), make pelleting by special molds, and at last obtain the soft capsules internally containing the tablet.

### Embodiment 6

This embodiment provides a vitamin K₂ (MK-7)-containing pharmaceutical composition for improving cardiovascular calcification, which includes, by weight parts, the following ingredients:

Vitamin K₂ (MK-7) as 0.3 parts, Ω-3 fatty acid as 1,500 parts, PQQ as 18 parts, coenzyme Q₁₀ (CoQ₁₀) as 180 parts, vitamin D₃ (VD₃) as 10 parts, soft capsule skin as 200 parts, enteric coating powder as 80 parts and auxiliary as 60 parts.

The preparation method is as follows:
(1) Granulating: Under the dark condition, mix MK-7, PQQ and tabletting auxiliary in equal proportion, and make granulation by dry method, where the particle size is controlled as 100 meshes, in order to obtain granulated particles;
(2) Tabletting: Use the tablet machine to make the particles obtained in Step (1) into naked tablets with a specified weight as 0.26 g;
(3) Coating tablet: Mix the naked tablets obtained in Step (2) with the enteric coating powder to obtain the coating tablets according to the normal coating steps;
(4) Capsule liquid: Weigh and take (2-3, CoQ₁₀, VD₃ and antioxidant by weight, dissolve the antioxidant, CoQ₁₀ and VD₃ into Ω-3 oil respectively at a temperature of 40° C. to obtain the mixed oil liquid, and then let it stand for 11 h to obtain the capsule liquid;
(5) Soft capsule skin: Obtain the soft capsule skins by adopting the general formula and preparation method in this industry;
(6) Soft capsule: Adopt the specialized equipment to inject the capsule liquid obtained in Step (4) between the two layers of capsule skins containing the coating tablet obtained in Step (3), make pelleting by special molds, and at last obtain the soft capsules internally containing the tablet.

### Verification Test

### 1. Investigation of Accelerated Stability of MK-7 Content in the Composition

Soft capsules prepared in Embodiments 4, 5 and 6 were respectively taken as the experimental group, and the commercial vitamin K₂ (MK-7) soft capsules (MK-7 content 90 μg) were purchased as the control group, all of which were individually sealed in brown bottles. Place them in a constant temperature and humidity box at 40±2° C. and 75%±5% RH environment, and do accelerated experiments. Samples were taken at 0, 1, 2, 3, 4, 5, 6, and 9 months to measure the MK-7 content. MK-7 content was determined by high performance liquid chromatography. The accelerated stability test results are as follows.

TABLE 1
Accelerated Stability Test

| | Time (Month) | | | | | | | |
|---|---|---|---|---|---|---|---|---|
| Content (%) | 0 | 1 | 2 | 3 | 4 | 5 | 6 | 9 |
| Embodiment 4 | 100.31 | 100.23 | 100.33 | 100.42 | 100.12 | 100 | 100.21 | 100.31 |
| Embodiment 5 | 100.49 | 100.42 | 100.41 | 100.28 | 100.24 | 100.37 | 100.32 | 100 |
| Embodiment 6 | 100.32 | 100.23 | 100.29 | 100.32 | 100.23 | 100.32 | 100.22 | 100.11 |
| Control group | 100.1 | 100 | 100.02 | 100.15 | 99.86 | 98.23 | 95.12 | 90.21 |

As evident from the results in Table 1, the MK-7 content in compositions from Embodiments 4, 5 and 6 remained largely unchanged, demonstrating stability, whereas the MK-7 content in the control group began to decline after 3 months of acceleration. It shows that in the pharmaceutical composition containing MK-7 prepared by the preparation method of the present disclosure, the active ingredient of MK-7 can remain stable for a long time. After calculation according to the normal accelerated conversion time, the shelf life can be guaranteed at least 3 years at room temperature.

### 2. Effect of Pharmaceutical Composition on Vascular Calcification in Rats

Select SPF grade SD male rats, healthy and 6 to 8 weeks old, with weight as 180 g-200 g.

Rats were randomly divided into normal control group (10 rats) and dexamethasone injection group (100 rats) according to body weight. The rats in the dexamethasone injection group were intramuscularly injected with dexamethasone 2.5 mg/kg three times a week, and the rats in normal control group were intramuscularly injected with a corresponding volume of normal saline. After 6 weeks of continuous injection, the injection was stopped. The rats were fasted for 24 hours, blood was taken from the orbital vein, and the serum was separated. A fully automatic biochemical analyzer was used to measure the alkaline phosphatase (ALP) level in rat serum. The ALP level of the rats in the dexamethasone injection group was compared with that of the rats in the normal control group to evaluate the modeling results. The rats that were successfully modeled were randomly divided into 5 groups: Model group, positive control group (pure product MK-7100 μg/kg), and experimental group (Embodiment 4, Embodiment 5, and Embodiment 6). The rats in normal control group and the model group were given 10 mg/kg edible oil. The rats in experimental group were given the corresponding MK-7 according to the established embodiment. The rats in positive control group were given MK-7 (12 mg/kg) dissolved in edible oil every day (once a day for 6 weeks). Rats in each group were housed in separate cages, with free access to food and water, and the room temperature was kept constant at (22±2° C.)

Observational indicators and methods: After the last dose, rats were fasted for 24 hours, blood was taken from the orbital vein, serum was separated, and OPG, OPN, CaBP, and BMP-7 levels in serum were measured using radioimmunoassay.

Determination of calcium content in the blood vessel: Dissolve 10 mg of abdominal aorta in nitric acid for digestion, dry it, and redissolve it with deionized water containing 27 nmol/L KCL and 27 μmol/L LaCl₃. After that, read the absorbency with an atomic spectrophotometer at 442.7 nm and convert it into calcium content in the tissue (μmol/mg·dw).

In terms of statistical methods, SPSS21.0 software was used for statistical analysis. Measurement data were expressed as mean±standard deviation. One-way analysis of variance and Dunnett-t test were used for analysis. P<0. 05 was considered a statistically significant difference.

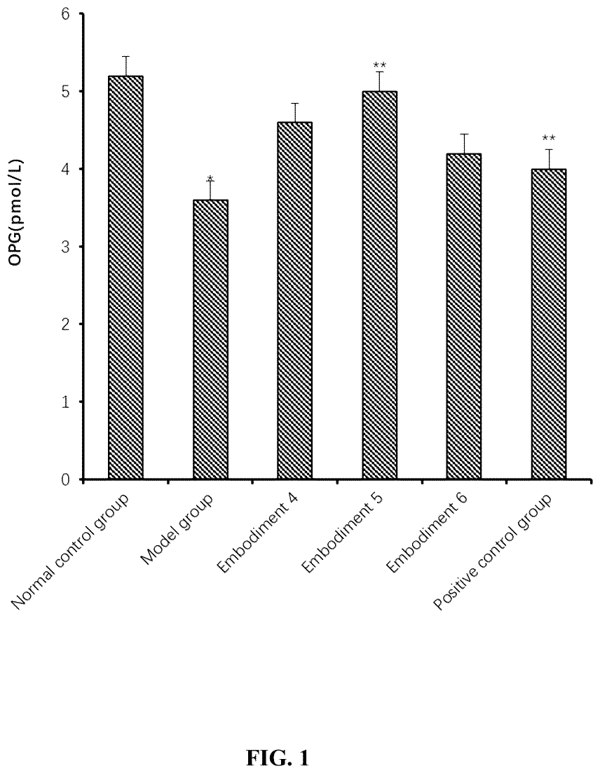

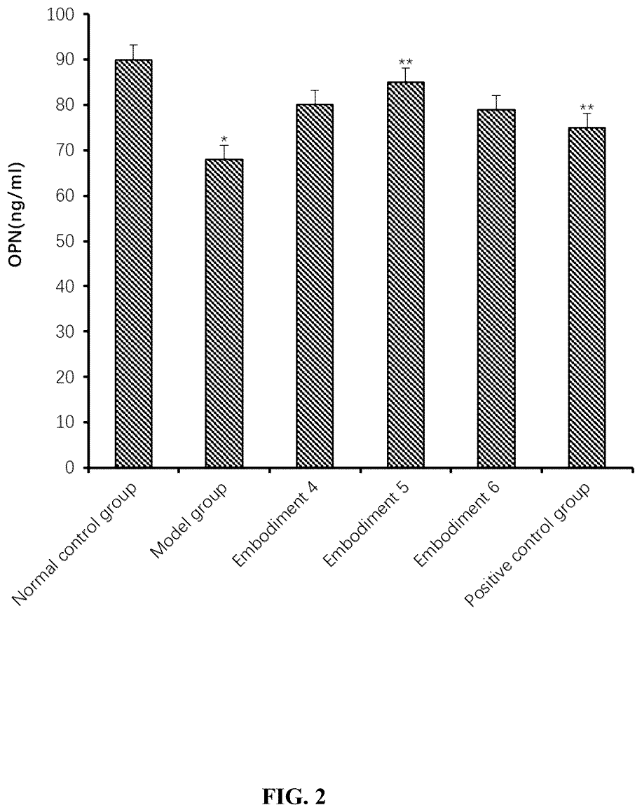

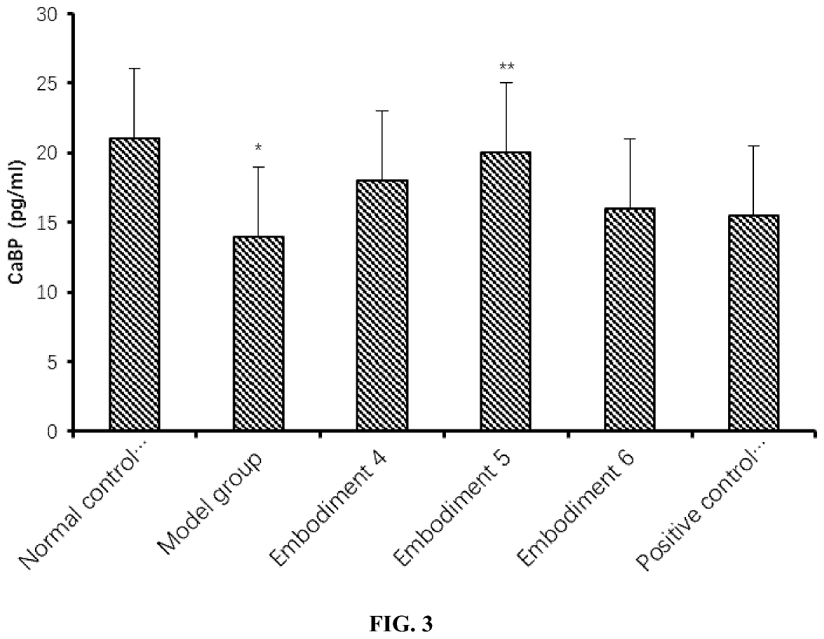

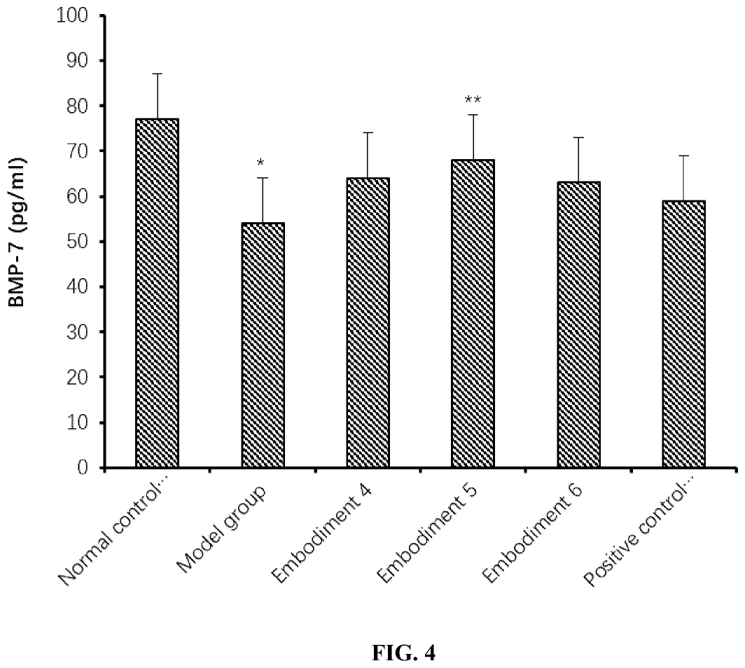

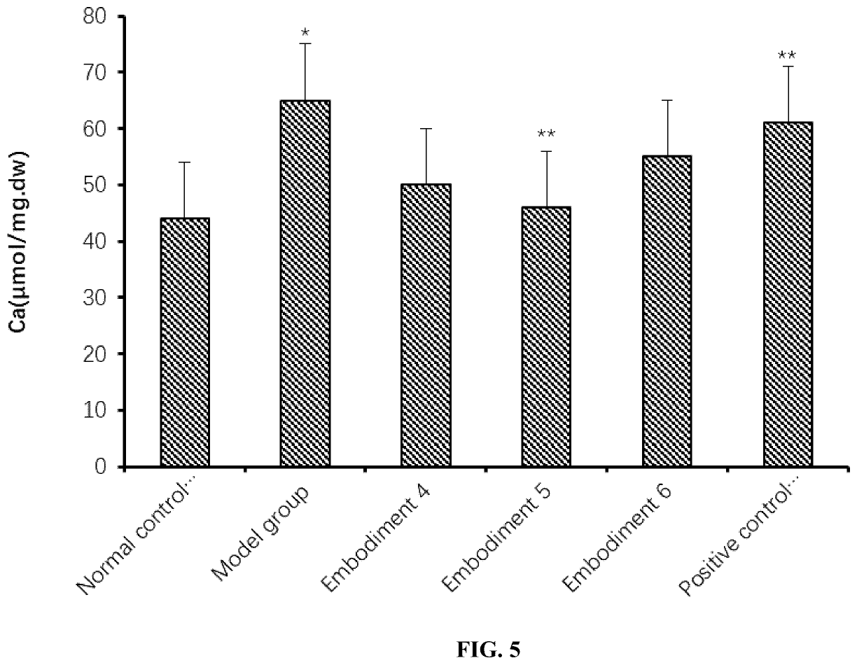

Combining with FIG. 1 to FIG. 5, we can see the effect of the pharmaceutical composition on vascular calcification in rats. Compared with the normal control group, the OPN (osteopontin), OPG (osteoprotegerin), CaBP (calcium binding protein) and BMP-7 (bone morphogenetic protein-7) levels in the serum of rats in the model group were significantly reduced; compared with the model group, the OPN (osteopontin), OPG (osteoprotegerin), CaBP (calcium binding protein) and BMP-7 (bone morphogenetic protein-7) levels in the serum of rats in the experimental group (Embodiment 4), experimental group (Embodiment 5) and experimental group (Embodiment 6) were significantly increased, with significant differences. At the same time, compared with the normal control group, the blood calcium level in the blood vessel of the rats in the model group was increased significantly; compared with the model group, the blood calcium levels in the blood vessel of the rats in the experimental group (Embodiment 4), experimental group (Embodiment 5) and experimental group (Embodiment 6) were significantly decreased, with significant differences.

OPN (osteopontin) is a marker gene for the transformation of vascular smooth muscle from a contractile phenotype to a synthetic phenotype, which can inhibit the calcification of cultured vascular smooth muscle cells; OPG (osteoprotegerin) can be expressed and released in vascular endothelial cells and smooth muscle cells, preventing the vascular damaging effects of inflammatory cytokines by increasing the survival of endothelial cells, and are related to atherosclerotic plaque rupture; as a bone morphogenetic protein, BMP-7 (bone morphogenetic protein-7) belongs to β family of transforming growth factors, which can improve vascular calcification by inhibiting the proliferation of vascular smooth muscle cells VSMC; calcium binding protein (CaBP) participates in cell cycle regulation, cell proliferation and differentiation, angiogenesis, extracellular matrix reconstruction and many other life processes, and has the effect of promoting the growth and survival for myocardial cells, which may promote the absorption of calcium and phosphorus in the upper section of small intestine and the release & transport of bone calcium.

In summary, the pharmaceutical composition of the present disclosure can regulate and inhibit the vascular calcification by significantly increasing the levels of OPN (osteopontin), OPG (osteoprotegerin), CaBP (calcium binding protein) and BMP-7 (bone morphogenetic protein-7), thereby reducing the calcium content in the blood vessel. Meanwhile, it was found through the experimental group and the positive control group that the pharmaceutical composition of the present disclosure has a compounded synergistic effect when compared with a single compound.

The above is only a preferred embodiment of the present disclosure, and is not intended to limit the scope of the present disclosure. Although the present disclosure has been disclosed in the above preferred embodiments, it is not intended to limit the present disclosure. Those skilled in the art can make some modifications or modifications to the equivalent embodiments by using the above-disclosed technical contents without departing from the technical scope of the present disclosure, but without departing from the technical solution of the present disclosure, according to the present disclosure. Any modification, equivalent change and modification of the above embodiments according to the technical substantials of the present disclosure are still within the scope of the technical solution of the present disclosure.

What is claimed is:

1. A vitamin K₂-containing pharmaceutical composition for improving a cardiovascular calcification, comprising a first component, a second component and a third component, wherein the first component comprises active functional ingredients for promoting calcium absorption, the second component comprises active functional ingredients for inhibiting or reversing calcification, and the third component comprises active functional ingredients for providing daily health protection for cardiovascular system; and
a mass ratio of the first component, the second component and the third component is (0.1-5): (0.01-0.5):(100-500).

2. The vitamin K₂-containing pharmaceutical composition for improving the cardiovascular calcification according to claim 1, wherein the first component comprises active ingredients for promoting calcium absorption, wherein the active ingredients for promoting calcium absorption comprise a main active ingredient and an auxiliary active ingredient, and the main active ingredient is vitamin D₃, and the auxiliary active ingredient is one or more selected from lactose, vitamin A, albumin polypeptide, vitellin peptide, fructooligosaccharide, galactooligosaccharide, and β-carotene.

3. The vitamin K₂-containing pharmaceutical composition for improving the cardiovascular calcification according to claim 1, wherein the second component comprises active ingredients for inhibiting or reversing calcification, wherein the active ingredients for inhibiting or reversing calcification comprise a main active ingredient and an auxiliary active ingredient, and the main active ingredient is vitamin K₂, and the auxiliary active ingredient is one or more selected from naringin, arachidonic acid, tripterine, puerarin, apigenin, and aconite polysaccharide.

4. The vitamin K₂-containing pharmaceutical composition for improving the cardiovascular calcification according to claim 1, wherein the third component comprises active ingredients for providing daily health protection for cardiovascular system, wherein the active ingredients for providing daily health protection for cardiovascular system comprise a main active ingredient and an auxiliary active ingredient, and the main active ingredient is Ω-3 fatty acid, pyrroloquinoline quinone, and coenzyme Q₁₀, and the auxiliary active ingredient is one or more selected from epicatechin, pumpkin seed oil, phospholipid, krill oil, spirulina, quercetin, lycopene, pomegranate polyphenol, resveratrol, cyanidin, soy isoflavone, and allicin.

5. The vitamin K₂-containing pharmaceutical composition for improving the cardiovascular calcification according to claim 1, wherein the first component accounts for 0.1%-1% based on a total mass of the pharmaceutical composition; the second component accounts for 0.001%-0.1% based on the total mass of the pharmaceutical composition; and the third component accounts for 30%-85% based on the total mass of the pharmaceutical composition.

6. The vitamin K₂-containing pharmaceutical composition for improving the cardiovascular calcification according to claim 1, further comprising an auxiliary, wherein the auxiliary is one or more selected from microcrystalline cellulose, silicon dioxide, magnesium stearate, α-cyclodextrin, mannitol, sodium carboxymethyl cellulose, hydroxy propyl cellulose, glycerol, gelatin, methyl cellulose, ethyl cellulose, hydroxypropyl methyl cellulose, talcum powder, modified starch, antioxidant, titanium dioxide, tartrazine aluminum lake, sunset yellow aluminum lake, carmine aluminum lake, brown iron oxide, glyceryl triacetate, polyethylene glycol, cross-linked povidone, and cross-linked sodium carboxymethylcellulose.

7. The vitamin K₂-containing pharmaceutical composition for improving the cardiovascular calcification according to claim 1, comprising the following main ingredients in respective parts by mass: 0.01-1 part of vitamin K₂, 300-3,000 parts of Ω-3 fatty acid, 1-40 parts of pyrroloquinoline quinone, 10-500 parts of coenzyme Q₁₀, and 1-60 parts of vitamin D₃.

8. The vitamin K₂-containing pharmaceutical composition for improving the cardiovascular calcification according to claim 1, wherein the vitamin K₂ is in a form of MK-7 with an all-trans structure; and
a ratio of EPA to DHA in the Ω-3 fatty acid is (0.1-4):1.

9. Use of the vitamin K₂-containing pharmaceutical composition for improving the cardiovascular calcification according to claim 1 in a dietary supplement and a health care product.

10. A soft capsule, comprising the vitamin K₂-containing pharmaceutical composition for improving the cardiovascular calcification according to claim 1.

11. The soft capsule according to claim 10, comprising the following main ingredients in respective parts by mass: 0.01-0.7 parts of vitamin K₂, 500-2,500 parts of Ω-3 fatty acid, 1-30 parts of pyrroloquinoline quinone, 50-500 parts of coenzyme Q₁₀, 1-50 parts of vitamin D₃, 50-500 parts of soft capsule skin, 10-100 parts of enteric coating powder, and 20-200 parts of an auxiliary.

12. A preparation method of the soft capsule according to claim 10, comprising the following steps:
(1) granulating: under a dark condition, evenly mixing fat-insoluble ingredients in the second component and the third component with some auxiliaries, followed by dry granulation, with a particle size being controlled at 50-200 meshes, to obtain granulated particles;
(2) tabletting: making the granulated particles obtained in the step (1) into naked tablets with a predetermined weight by using a tablet machine;
(3) coated tablet: mixing the naked tablets obtained in the step (2) with an enteric coating powder to obtain coated tablets according to normal coating steps;
(4) capsule liquid: weighing fat-soluble ingredients in the third component, the first component and an auxiliary of an antioxidant, dissolving the antioxidant and the first component into an oil with the fat-soluble ingredients in the third component separately at a temperature of 20-60° C. to obtain a mixed oil solution, and then allowing the mixed oil solution to stand for 4-12 hours to obtain the capsule liquid after defoaming;
(5) soft capsule skin: obtaining the soft capsule skin by using a general formula and preparation method; and
(6) soft capsule: injecting the capsule liquid obtained in the step (4) between two layers of the soft capsule skin containing the coated tablet obtained in the step (3), followed by pelleting to obtain the soft capsule containing the coated tablet therein.

13. A preparation method of the soft capsule according to claim 11, comprising the following steps:
(1) granulating: under a dark condition, evenly mixing fat-insoluble ingredients in the second component and the third component with some auxiliaries, followed by dry granulation, with a particle size being controlled at 50-200 meshes, to obtain granulated particles;
(2) tabletting: making the granulated particles obtained in the step (1) into naked tablets with a predetermined weight by using a tablet machine;
(3) coated tablet: mixing the naked tablets obtained in the step (2) with an enteric coating powder to obtain coated tablets according to normal coating steps;
(4) capsule liquid: weighing fat-soluble ingredients in the third component, the first component a and the auxiliary of an antioxidant, dissolving the antioxidant and the first component into an oil with the fat-soluble ingredients in the third component separately at a temperature of 20-60° C. to obtain a mixed oil solution, and then allowing the mixed oil solution to stand for 4-12 hours to obtain the capsule liquid after defoaming;
(5) soft capsule skin: obtaining the soft capsule skin by using a general formula and preparation method; and
(6) soft capsule: injecting the capsule liquid obtained in the step (4) between two layers of the soft capsule skin containing the coated tablet obtained in the step (3), followed by pelleting to obtain the soft capsule containing the coated tablet therein.
